(19) United States
(12) Patent Application Publication
(10) Pub. No.: US 2024/0023883 A1
(43) Pub. Date: Jan. 25, 2024

(54) METHOD AND DEVICE FOR EVALUATING MUSCLE DEVELOPMENT IN AN ANIMAL

(71) Applicant: CAN TECHNOLOGIES, INC., Wayzata, MN (US)

(72) Inventor: Abby KEEGAN, Mount Vernon, IA (US)

(73) Assignee: CAN TECHNOLOGIES, INC., Wayzata, MN (US)

(21) Appl. No.: 18/362,539

(22) Filed: Jul. 31, 2023

Related U.S. Application Data
(63) Continuation of application No. 16/631,096, filed on Jan. 14, 2020, now abandoned.
(60) Provisional application No. 62/532,756, filed on Jul. 14, 2017.

(51) Int. Cl. A61B 5/00; G01B 3/02; G06T 7/00; G06T 13/40

(52) U.S. Cl. CPC A61B 5/4519; G01B 3/02; G06T 7/0014; G06T 13/40; A61B 2503/40; G06T 2207/30004

(57) ABSTRACT

The present invention relates to methods and devices for measuring the musculature of animal. In some embodiments, the musculature of the animal is measured using by generating traces of the animal using a flexicurve ruler and comparing the traces to a reference and/or previous traces of the animal. The methods can be used to improve an animal's musculature by evaluating the muscle development of the animal and changing the animal's diet or exercise regime based on the results of the evaluation.

### CROSS-REFERENCE TO RELATED APPLICATIONS

This application is a continuation of U.S. patent application Ser. No. 16/631,096, filed Jan. 14, 2020, which is a national phase application of International Application No. PCT/US2018/042276, filed Jul. 16, 2018, which claims the benefit of U.S. Provisional Patent Application No. 62/532,756, filed Jul. 14, 2017, each of which is hereby incorporated by reference in its entirety.

### BACKGROUND

The evaluation of a horse's topline, which surrounds and supports the spine, is of interest to horse owners, equine veterinarians, chiropractors, nutritionists, and researchers to gauge changes in muscle condition. However, it can be difficult to accurately measure and evaluate changes in muscling using present methods, for example via photos and other subjective methods such as Topline Evaluation Scoring. Ultrasound is a useful tool, however it is limiting due to availability, high expense, and user training. The horse industry generally does not understand the influence of nutrition on building and maintaining muscle. A reliable method for evaluating and tracking muscle development in a horse or other animal could be useful for nutritionists and veterinarians to aid in understanding about the effects of nutrition on muscle supporting and surrounding the spine, as well as to aid in designing and implementing diets to improve muscle condition.

### SUMMARY

A method and tool or system for evaluating muscle condition and development in an animal is described. In one aspect, the method is a method for evaluating muscle development in the topline of a horse, comprising: generating one or more evaluation traces of a horse's topline, wherein the evaluation traces are generated by molding a flexible measurement tool to one or more locations of the horse's topline, calculating the area under the curve of the one or more evaluation traces, and comparing the area under the curve of the one or more evaluation traces to the area under the curve of one or more reference traces. In one aspect, the method is a method for evaluating an attribute of an animal, comprising: obtaining one or more reference images of a location of an animal, wherein the one or more reference images are obtained via a 3D scanner; obtaining one or more evaluation images of the same location of the same animal, wherein the one or more evaluation images are obtained via a 3D scanner; measuring a value of the attribute of the location of the animal from the one or more reference images; measuring a value of the attribute of the same location of the same animal from the one or more evaluation images; and comparing the value of the attribute measured in the one or more evaluation images with the value of the attribute measured in the reference images.

In some embodiments, the one or more evaluation traces comprise a trace of the horse's croup, loin, and/or wither. In some embodiments, the one or more reference traces is a trace of the same horse's croup, loin, and/or wither generated on day 0. In some embodiments, the reference trace is a previous evaluation trace of the same horse's croup, loin, and/or wither generated at a time between day 0 and the time the evaluation trace of the horse's topline is generated. In some embodiments, if the area under the curve of the evaluation trace is greater than the reference trace, the musculature of the horse has improved since the time the reference trace was generated.

In some embodiments, the method further comprises changing a care characteristic of the horse if the curve of the evaluation trace is less than the reference trace. In some embodiments, the care characteristic is the horse's diet. In some embodiments, the method further comprises weighing the animal. In some embodiments, the method further comprises generating a Topline Evaluation Score (TES) from the one or more evaluation traces and/or the one or more reference traces.

In some embodiments, the method further includes determining the change in the value of the attribute. In some embodiments, the method further includes the step of changing the animal's diet or changing some other care characteristic of the animal based on change in the value of the attribute. In some embodiments, the method further includes administering a diet and/or a care characteristic between the time of the reference trace, image, or scan and the time of the evaluation trace, image, or scan.

In one aspect, the flexible measurement tool is a flexicurve tool or ruler. In some embodiments, the flexible measurement tool comprises a midpoint reference marking for positioning the center of the tool above the horse's spine. In some embodiments, the flexible measurement tool comprises left and right reference markings for aligning the tool with the left and right sides of the horse. In some embodiments, a 3D scanner is used instead of a flexible measurement tool.

In one aspect, the system is a system for measuring the musculature of an animal, comprising: a flexible measurement device, wherein the measurement device comprises a material suitable for molding into a shape corresponding to an anatomical location of an animal and also suitable for maintaining the shape of the molding when the device is not in contact with the animal, wherein the measurement device comprises a marking for aligning the center of the measurement device with the spine of an animal and markings for specifying the left and right side of the measurement device, and a tracing device for generating a tracing of the shape of the flexible measurement device. In some embodiments, the system further comprises a device for comparing the area under the curve of two or more tracings generated by the tracing device. In some embodiments, in the tracing device is a camera suitable for imaging the shape of the measurement device. In some embodiments, the system further comprises instructions for measuring one or more anatomical locations useful for measuring the musculature of an animal.

It is also to be understood that the elements or aspects of any embodiment of the processes, methods, or compositions described above can be applied to any other embodiment, as would be understood by a person skilled in the art. For example, a measurement of an anatomical part of an animal obtained from a scan or image obtained from a 3D scanner can be substituted for the tracings obtained the flexible measurement tool.

### BRIEF DESCRIPTION OF THE DRAWINGS

The following detailed description of the invention will be better understood when read in conjunction with the appended drawings. It should be understood, however, that the invention is not limited to the precise arrangements and instrumentalities of the embodiments shown in the drawings.

FIG. 1 is an image showing tracings obtained by molding a flexicurve tool to a horse at three different locations (croup, loin, and wither) then removing the tool while maintaining the molded shape and tracing the molded tool. The area under the curve increased from day 0 (lower lines for each location) to day 9 (upper lines for each location), therefore indicating an increase in lean muscle.

FIG. 2 is a photo image of a horse with a flexicurve tool (101) molded to a location of interest for measuring topline musculature.

FIG. 3 is a diagram of an exemplary embodiment of a device for evaluating muscle development in an animal.

FIG. 4, comprising FIGS. 4A, 4B, and 4C, is a series of photos images of a horse fed a trial diet to improve topline muscling after an initial diet of hay only. The horse's topline muscle development was evaluated using an exemplary embodiment of a device and method described herein. 4A=measurement at day 0, hay diet only, Body Condition Score (BCS)=6, Topline Evaluation Score (TES)=C; 4B=measurement after 9 weeks on trial diet, BCS=6, TES=B; 4C=measurement after 12 weeks on trial diet, BCS=6, TES=A. The horse in FIG. 4 shows an improvement in TES with no change in BCS as a result of the trial diet using Nutrena™ Empower Topline Balance™ supplement available from Cargill.

FIG. 5, comprising FIGS. 5A through 5C, is a series of photos of a flexible measurement tool (101) molded to a horse at three different locations useful for a topline muscle evaluation.

FIG. 6 is an image of a horse obtained using a 3D scanner.

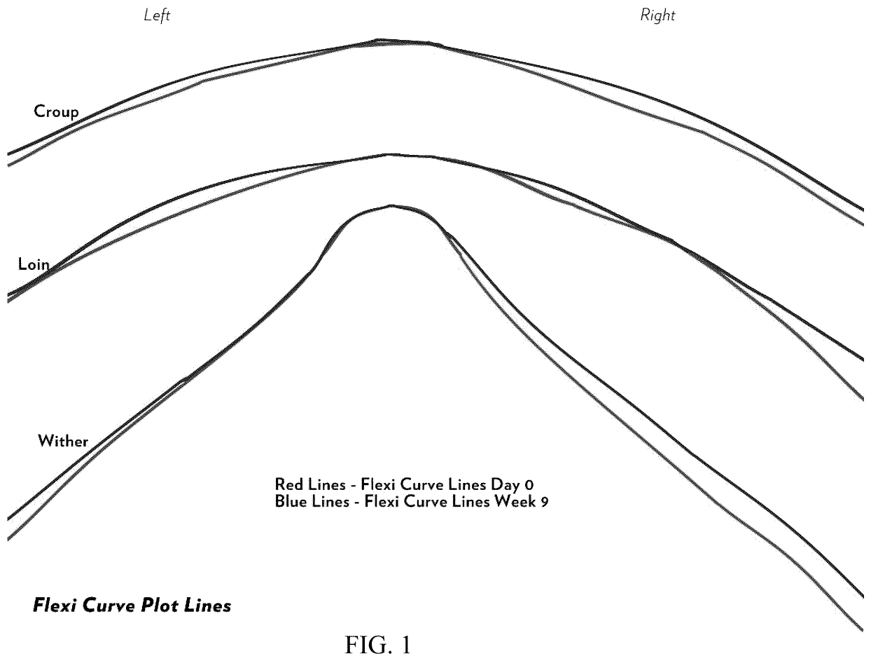

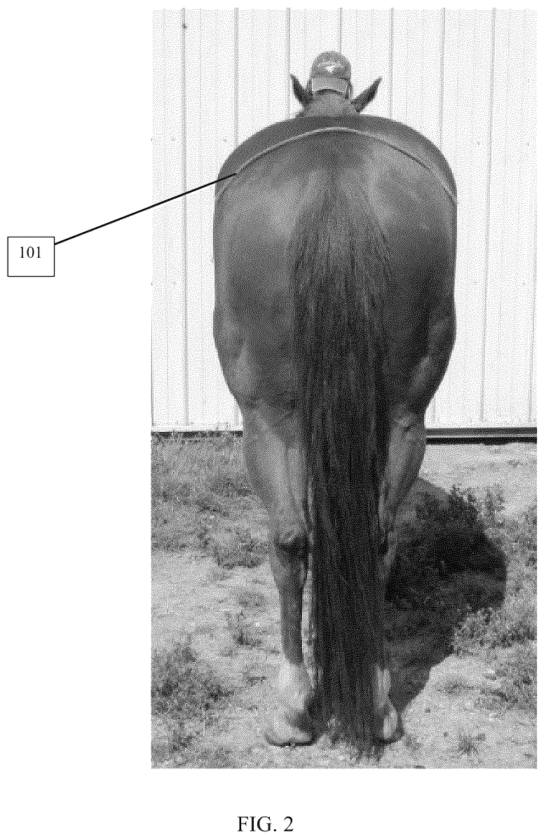

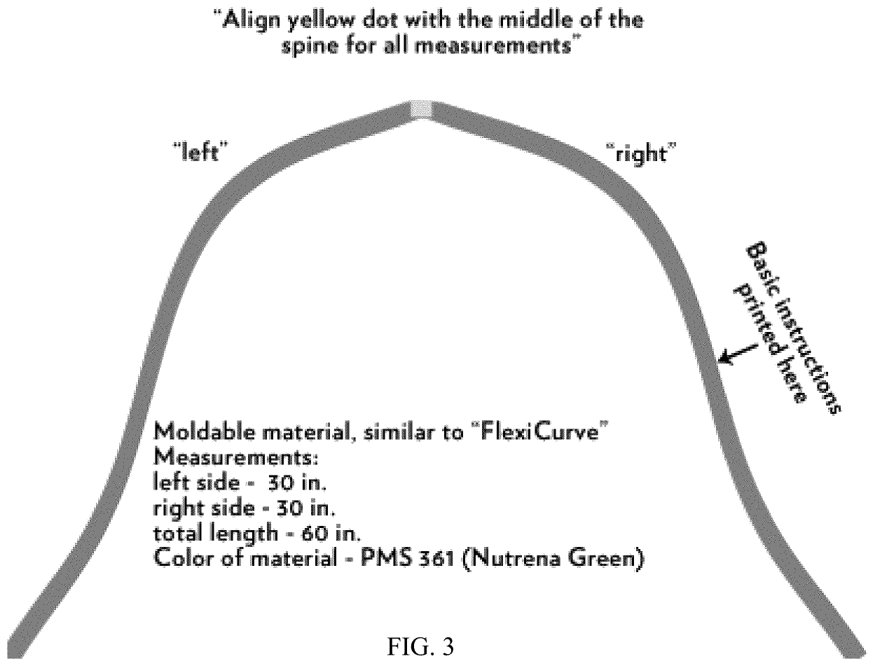

### DETAILED DESCRIPTION

It is to be understood that the figures and descriptions of the present invention provided herein have been simplified to illustrate elements that are relevant for a clear understanding of the present invention, while eliminating other elements found in the related field(s) of art. Those of ordinary skill in the art would recognize that other elements or steps may be desirable or required in implementing the present invention. However, because such elements or steps are well known in the art or do not facilitate a better understanding of the present invention, a discussion of such elements or steps is not provided herein.

Unless defined otherwise, all technical and scientific terms used herein have the same meaning as commonly understood by one of ordinary skill in the art to which this invention belongs. As used herein, each of the following terms has the meaning associated with it as defined in this section.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 7 should be considered to have specifically disclosed subranges such as from 1 to 3, from 1 to 4, from 1 to 6, from 2 to 5, from 3 to 5, etc., as well as individual numbers within that range, for example, 1, 2, 3, 3.6, 4, 5, 5.8, 6, 7, and any whole and partial increments in between. This applies regardless of the breadth of the range.

### Methods for Evaluating and Improving Musculature of an Animal

Described herein are methods for evaluating changes in the musculature of an animal by tracing or otherwise measuring a portion of the animal. The methods can further include the aspect of improving the musculature by changing the diet or other care characteristic of the animal based on the musculature evaluation. In one aspect, the method is useful for evaluating the topline of a horse. In some embodiments, the tracing is performed using a flexicurve ruler or other suitable device that can be molded to the contours of an animal's body. The tracing can be performed on specific areas of the animal and these areas can be molded and traced again at the same location at a later time to evaluate change. In one aspect, the tracing is performed using a 3D scanner or sensor to generate images of a specific area of the animal.

It is commonly thought in the horse industry that significant improvements in topline muscle can only be achieved through changes in work or exercise and not through changes in diet, especially changes in diet alone. In one aspect, the method and tool described herein are useful for quantitatively and/or qualitatively evaluating changes in topline musculature, for example to demonstrate the effects of dietary changes. Using the method described herein, it has been shown that a horse's topline evaluation score (TES) can improve based on changes in diet alone, and that such an improvement can be achieved with little or no change to the horse's body condition score (BCS).

In some embodiments, the method for evaluating the musculature of an animal includes the steps of: positioning a flexible measurement tool on the animal such that the center point of the tool is positioned on the midline of the animal's spine and the sides of the tool extend to both the left and right sides of the animal; molding the flexible measurement tool around the muscle surrounding the spine on each side of the animal; after the measurement tool has been closely molded to the shape of the animal, removing the tool from the animal while maintaining the molded shape; generating a tracing of the molded measurement tool (the evaluation tracing); and comparing the evaluation tracing of the molded measurement tool to a reference tracing. Non-limiting examples of devices suitable for use as the flexible measurement tool are described in more detail elsewhere herein. In some embodiments, the evaluation tracing and reference tracing can be compared by calculating the area under the curve of each tracing.

The method can include the measurement of a single location on the animal, or it can include the measurement of multiple locations. As would be understood by a person skilled in the art, measuring multiple locations can result in a more accurate and quantitative measurement of changes in musculature in the animal. Further, a skilled artisan can readily determine the location or locations that are most suitable for measurement, depending on the species of animal and musculature of interest.

By way of example, the topline musculature of a horse can be evaluated by measuring the following locations: croup, loin, and/or wither. Croup can be measured one hand's length (approximately 7″) up from top of the horse's tailhead with the mid-point of the measurement tool aligned with middle of spine and left and right sides positioned to corresponding sides of the horse. Loin can be measured by palpating the last rib and following the rib curve up to the top of the spine, aligning the tool with the middle of the spine and molding the left and right sides of the tool to the horse accordingly. Wither can be measured by placing the mid-point of the tool on the middle of the spine at the base of the mane (i.e., where the hair ends) and molding the left and right sides of the tool to the horse accordingly.

In some embodiments, the method can be used to generate quantitative and qualitative evidence of compositional changes in body mass of mature horses, specifically fat-free mass shifts (lean muscle tissue) in relation to plane of nutrition. For example, in addition to croup, loin, and wither, as described above, the back can also be measured by palpating the 17th and 18th ribs, placing the measurement tool parallel to the ribs in the space between the two ribs with the midpoint located on top of the spine and molding the left and right sides of the tool to the horse accordingly.

The method described herein is not limited to the topline of a horse, but can be performed on any species of animal and any anatomical location on an animal. Other animals include, but are not limited to cattle, pigs, poultry, and lambs.

The methods described herein include a step of generating a tracing of the measurement tool after it has been molded to an anatomical location on an animal. In some embodiments, the molded measurement tool is removed from the animal and placed on a piece of paper, for example graph paper, and manually traced. However, the method of generating the tracing of the measurement tool is not limited to manual tracing. In some embodiments, the tracing step can be performed using image-recognition software implemented via a computer. For example, a mobile phone or tablet computer with a camera can be used to capture the tracing of the molded measurement tool via a photo image. The photo image of the tracing can be stored and compared to later-generated tracings of the same anatomical location on the animal. Further, the area under the curve (AUC) of the tracing can be automatically calculated using software methods known in the art. For example, a software program such as CAD can be used to determine AUC and compare the before and after changes in lean muscle tissue.

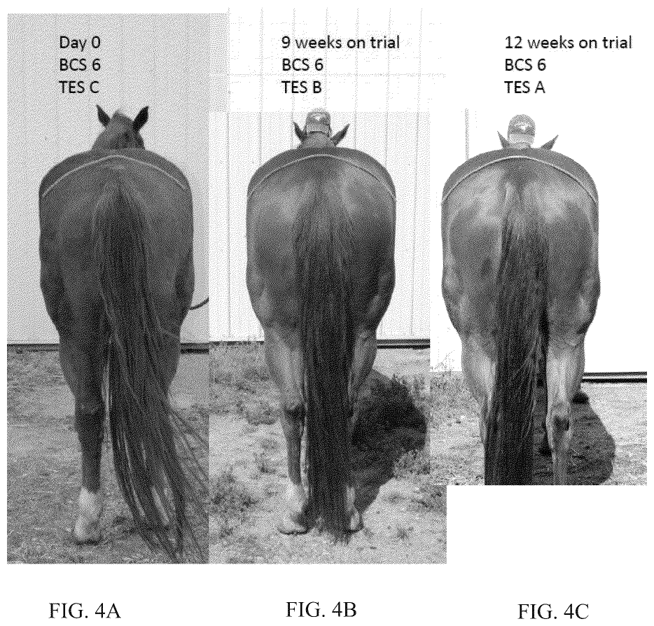

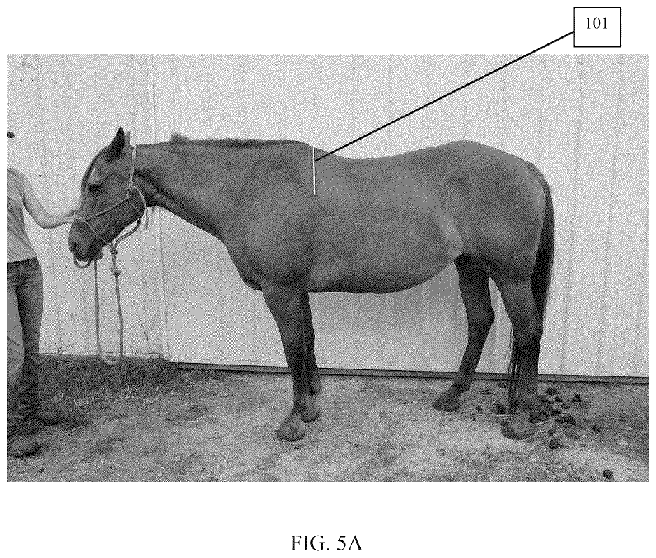

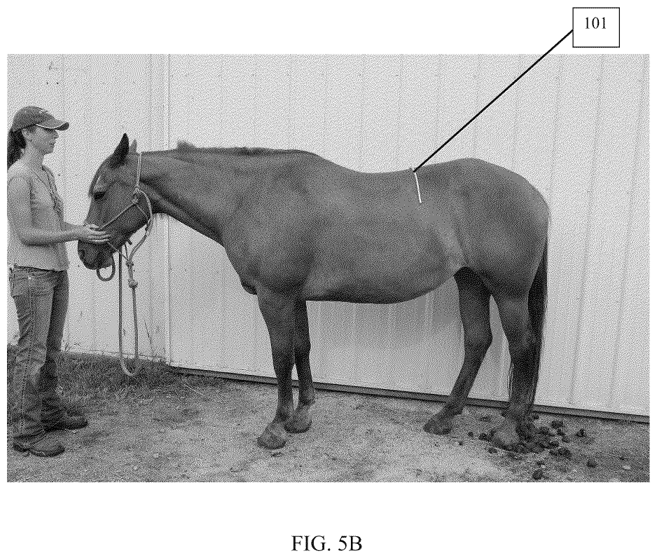

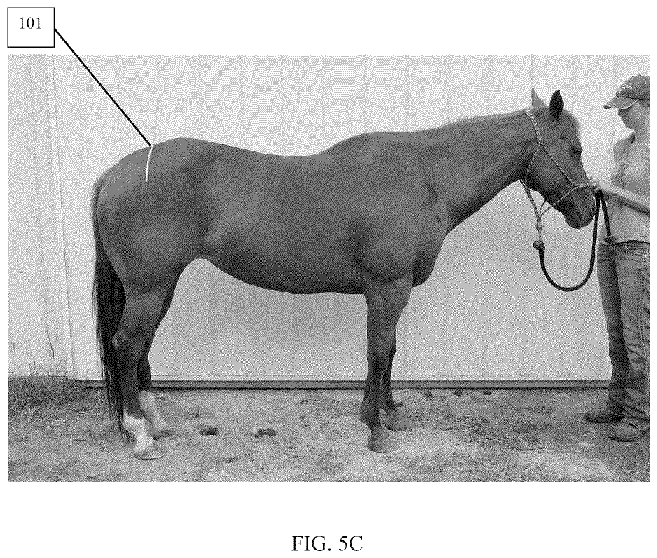

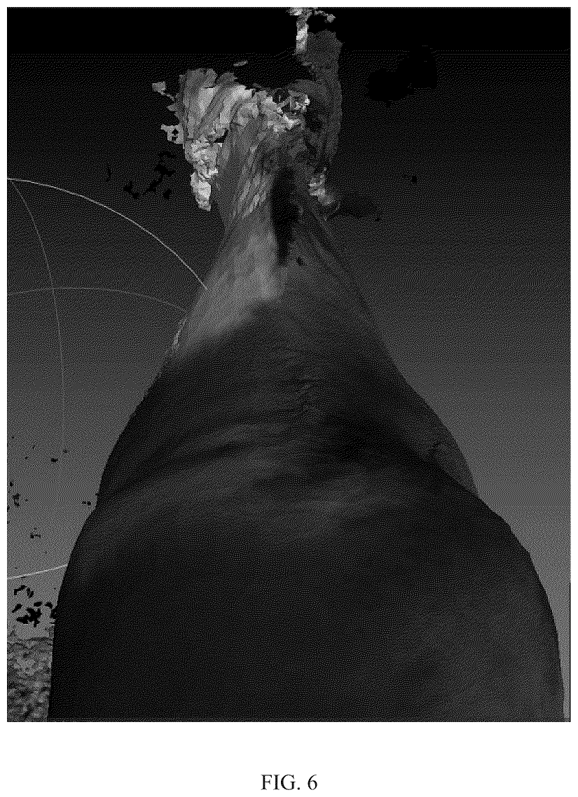

In one aspect, the method can be performed using a 3D scanner. In such embodiments, the 3D scanner can be used to acquire three-dimensional images or scans of an animal which can be used to evaluate muscle development in the animal. These 3D images or scans can be used as the tracings described in other embodiments of the method described herein (see, e.g., FIG. 6, which is an image of a horse obtained with a 3D scanner that shows a comparable location and view of the horse compared to an image obtained with a camera as shown in FIG. 4). Accordingly, in some embodiments, a software program can be used to measure the desired shape and/or size of a location of an animal, for example a horse's topline, from a 3D image or scan. As would be understood by a person skilled in the art, the embodiment of the method using a 3D scanner can include any of the other steps or aspects of the method which is performed using a flexible measurement tool.

3D scanner technology is well known in the art. Software useful for acquiring scans using the scanner and analyzing the acquired scans is also well known in the art. The 3D scans obtained using the scanner may also be referred to as volumetric scans. An example of a commercially available 3D scanner is the Structure Sensor available from Occipital. A variety of software applications which can be used to capture and/or analyze 3D scans from the scanner are available commercially (e.g., the Structure v.1.4 application by Occipital). The software can be used with a suitable microprocessor system, such as an Apple iPad™ tablet computer. An example of an apparatus for 3D scanning which can be used with the methods described herein is provided in U.S. Patent Application Publication No. US 2015/0077517 (Powers et al.) published on Mar. 19, 2015, which is hereby incorporated by reference in its entirety.

In some embodiments, the method is a method for evaluating an attribute of an animal, for example the muscle condition and/or muscle development in the animal, and can include the steps of: obtaining one or more reference images of a location of an animal, wherein the one or more images are obtained via a 3D scanner or 3D sensor system; obtaining one or more evaluation images of the same location of the same animal, wherein the one or more evaluation images are obtained via a 3D scanner or 3D sensor system; measuring a value of an attribute of the location of the animal from the one or more evaluation images; measuring a value of the same attribute of the same location of the animal from the one or more reference images; and comparing the value of the attribute measured in the one or more evaluation images with the value of the attribute measured in the reference images. In some embodiments, the method further includes determining the change in the value of the attribute. In some embodiments, the method further includes the step of changing the animal's diet or changing some other care characteristic of the animal based on change in the value of the attribute. In some embodiments, the evaluation image(s) are obtained after administering a diet or feed composition to the animal for a period of time, such that the comparison of the evaluation image(s) to the reference image can indicate the effects of administering the diet or feed composition to the animal. In some embodiments, the evaluation image(s) are obtained after changing a care characteristic of the animal for a period of time, such that the comparison of the evaluation image(s) to the reference image can indicate the effects of changing the care characteristic of the animal.

In some embodiments, the location of the animal is a horse's topline. In some embodiments, the attribute is the shape of the location of the animal. In some embodiments, the attribute is the size of the location of the animal, or the size of a specific aspect of the location. In some embodiments, such a size can be the area of a two-dimensional cross-section of the location, or it can be the volume of a three-dimensional shape of the location. In one aspect, the attribute can be any metric or characteristic associated with an animal's health or well-being.

It is to be understood that the phrase “value of an attribute” can also mean “value associated with an attribute.” In some embodiments, the value of the attribute can be quantitative, for example the area under the curve of the topline of the horse as previously described. In some embodiments, the value of the attribute can be qualitative, for example a rating or score associated with the shape of the topline of the horse. In some embodiments, the value of the attribute, whether it is a quantitative or qualitative value, can be measured or determined automatically via computer software.

Use of a 3D scanner or sensor to capture the images used in the method can provide advantages over use of a flexible measurement tool. Such advantages, include but are not limited to: avoiding user error, for example inconsistency in the level of pressure applied to the flexible tool when measuring the animal; avoiding inconsistency or inaccuracy in transferring the shape of the flexible tool to a tracing; avoiding the need to physically touch the animal; and reduction in time needed to make the measurements.

In addition, the use of a 3D scanner can be used to capture more data and/or different types of data than the use of the flexible measurement tool in the method. In one aspect, the 3D scanner can be used to create a 3D model of the animal or a specific location of the animal. Accordingly, the method can also include the step of generating a 3D model of an animal or portion of the animal. Such a 3D model can be used to measure the volume of the portion of the animal, which would be difficult or impossible using two-dimensional images or tracings.

For the purposes of evaluating muscle development in an animal, it is to be understood that the method includes a step of comparing the tracing of the molded measurement tool (the evaluation tracing) to a reference tracing. In some embodiments, the reference tracing is a tracing obtained in the same manner and on the same animal as the evaluation tracing, but at an earlier time. For example, an animal can be measured using the method described herein at day 0 and again at a later time, for example day 7. The day 0 tracing is used as the reference tracing and the day 7 tracing is the evaluation tracing. Changes in the area under the curve (AUC) of the tracing can be used to evaluate changes in the animal's musculature. An increase in the AUC can indicate an increase in lean muscle mass. One or more evaluation tracings can be obtained at regular or irregular intervals over any period of time, including days, weeks, or months. As described above, a 3D scan or image can be obtained and used to generate the tracing.

In some embodiments, the reference tracing can be a tracing that was not generated from the animal to be evaluated. In some embodiments, the reference tracing can be an average of multiple tracings generated from two or more animals. As would be understood by a person skilled in the art, the method described herein can be used to generate tracings for comparing two different animals in addition to comparing changes in a single animal.

The method can include other aspects or steps. In some embodiments, the musculature evaluation is used to determine additional steps for the animal's care. Such additional steps can include modifying the animal's diet or modifying the animal's exercise or physical activity regimen. In some embodiments, the method also includes the step of weighing the animal.

### Devices and Systems for Musculature Evaluation

Described herein are flexible measurement devices useful for evaluating animal musculature. In some embodiments, the device includes markings for orienting the device on the appropriate portion of the animal. Use of the markings ensure that the device is applied to the animal so that a series of measurements taken over time using the device are consistent and accurate. Further, the device is suitably flexible for molding to the contours of an animal's body, but is also suitably rigid to maintain the molded shape once removed from the animal.

In some embodiments, the device can be any tool of suitable length and flexibility for molding to a portion of an animal's body. In some embodiments, the device is a strip or cord of flexible material, such as a flexible polymer, rubber, or plastic. In some embodiments, the device includes a core that is more rigid than the outer material surrounding the core, for example a metal core that is coated with or enveloped by a more flexible material. Such a rigid core can assist in maintaining the shape of the measurement tool after it is molded.

In some embodiments, the flexible measurement device is a flexicurve ruler or tool. For the purposes of this disclosure, “flexicurve,” “flex-curve,” “flexible curve” are used interchangeably, and refer to any strip of flexible or malleable material that can substantially maintain a shape after being bent, molded, or otherwise re-shaped. A non-limiting example of a flexicurve is the Quint Measuring Systems™ FC36 flexible curve ruler, which is a commercially available product. In some embodiments, the flexible measurement device includes markings to assist in proper orientation of the device on the animal, for example, a center marking for alignment with the animal's spine and left/right markings for consistent alignment with the animal's left and right sides.

In some embodiments, the measurement tool can include electronics associated with positioning, for example sensors or gyroscopes, that can be used to generate the tracing automatically. For example, the size, shape, and position of all segments of the entire measurement tool can be determined electronically once the measurement tool is molded to the animal, without the need for removing the tool and tracing separately. Sensors in the tool can be used to aid in the positioning and molding of the tool to the animal, to increase the consistency and accuracy of the measurements. Sensors can also be used to determine the position of different segments of the measurement tool in relation to each other, to elucidate the shape of the molded measurement tool and therefore generate a tracing. The measurement tool can be connected to a computer or mobile device via wire and/or wirelessly so that an electronic image or diagram of the measurement tool can be transmitted and stored as necessary. In some embodiments, the measurement tool can be a software-based tool, for example, the measurement tool can include a suitable camera and software that recognizes the location and shape of a tracing on the animal without the need for physically molding a flexible measurement tool to the animal.

Described herein is also a system for measuring the musculature of an animal which includes the flexible measurement tool. The system further includes a device for generating a tracing of the shape of the flexible measurement tool after it is molded to the shape of an anatomical part of an animal. The device for generating the tracing can be an imaging device, such as a mobile phone equipped with a camera, or it can be a manual tracing tool such as paper combined with a writing utensil. The system further includes instructions for positioning the measurement tool appropriately on the animal, for generating tracings of the selected anatomical portion of the animal (both evaluation and reference tracings), for calculating the AUC of the tracings, and/or for making an evaluation of the musculature of the animal based on comparing an evaluation tracing and a reference tracing.

In some embodiments, the system for measuring the musculature of an animal includes a 3D scanner or a camera, a microprocessor system suitable for receiving a scan or an image from the 3D scanner or camera, and a software program suitable for identifying a location on an animal and measuring a value of an attribute of the location from the scan or the image. As would be understood by a person skilled in the art, the system can include any other hardware or software needed to perform the method, such as storage or access to storage for storing scans or images, and hardware or software suitable for processing the images according to the method.

It is to be understood that any of the aspects or embodiments for the flexible measurement tool or device described herein can be applied for use in the methods and systems for evaluating muscle development of the present invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

### Example 1: Horse Feeding Trial

A horse feeding trial was conducted to evaluate the effects of a change in feed using the tool and method described herein. Three horses from the same farm were fed a good quality hay only diet prior to the feeding trial. A hay only diet for horses is very common amongst horse owners. The horses all had good health and were up-to-date in vaccinations and worming at the time of the trial. The objective of the trial was to demonstrate that by properly balancing any missing nutrients in the hay, the horses would show an improvement in lean muscle tissue surrounding and supporting the spine while maintaining a similar Body Condition Score (BCS) (a measure of fat deposition on the horse). The improvement in Topline Evaluation Score (TES) (or lack thereof) was evaluated using an embodiment of the tool and method described herein. All three horses had no change in work during the trial period. All three horses remained on the same hay during the trial period, which was supplemented with the same feed supplement for each horse, and did not have access to any additional forage. During the trial, horses were placed on 0.03% of their bodyweight of feed supplement (per feeding directions on tag) and the total pounds were divided into two meals per day. The feed supplement was Nutrena™ Empower Topline Balance™ supplement available from Cargill.

Embodiments of a method and device for measuring horse musculature were used during a horse feeding trial. Tracings were generated by molding a flexicurve tool with a marking for the midline and markings for designating the left and right sides of the horse's spine to provide proper orientation of the flexicurve tool on the horse. Tracings were generated for three locations (croup, loin, and wither) on each of three horses. Each set of tracings was repeated for each horse at different time points. A handler ensures each horse is standing in all three legs and is relaxed prior to each tracing and holds the horse for safety of the measurer. The measurer places the flexicurve over the desired location on the horse, molds the flexicurve to the horse's body at that location while ensuring proper orientation of the flexicurve, then removes the flexicurve while taking care to maintain the shape of the molded flexicurve. The molded flexicurve is then placed on graph paper and the area under the curve (AUC) is measured using the graph paper.

The feeding regimen for each horse and weight measurements are shown in Table 1 below. Increases in AUC for the flexicurve measurements corresponded to increases in weight and lean muscle tissue. The trial successfully demonstrated that the method and device of the present invention can be used to measure and track changes in lean muscle tissue on the topline of a horse that correlate with subjective Topline Evaluation Scoring (A-D, where A is ideal muscling in all three areas and D is inadequate muscling in all three areas of the topline). The trial also demonstrated that the horses showed little or no change in BCS, but showed significant improvement in TES. The AUC measured using the method also correlated with subjective TES scores with respect to observed improvements in topline muscling.

TABLE 1

| | | Trial Feeding | | | | | | | | | | Increase in |
|---|---|---|---|---|---|---|---|---|---|---|---|---|
| | | Program | | | | | | | | | | AUC via |
| Horse | | (Product's & #/day, | | | | | | | | | | Flexi- |
| Name, | Diet | supplement | | Day 30 | Day 69 | | | | | | | Curve |
| Breed, & | prior | amounts, % bwt in | Day 0 | scale | scale | Day 0 | Day 30 | Day 69 | Day 0 | Day 30 | Day 69 | method |
| Description | to trial | hay/day) | weight | wt | wt | BCS | BCS | BCS | TES | TES | TES | tracings |
| Dynah 17 | hay | 3# Empower | 71.75/ | 1045 | 1025 | 6 | 6 | 6 | C | B | B | YES |
| year old | only | TLB/day | 65 = | | | | | | | | | |
| AQHA | | | 1014 | | | | | | | | | |
| Mare | | | | | | | | | | | | |
| (chestnut) | | | | | | | | | | | | |
| Chance 18 | hay | 3.5# Baxpower | 75/68 = | 1150 | 1130 | 5 | 5 | 5.75 | D | C | B+ | YES |
| year old | only | TLB/May | 1159 | | | | | | | | | |
| AQHA | | | | | | | | | | | | |
| Gelding | | | | | | | | | | | | |
| (bay) | | | | | | | | | | | | |
| Roxy aged | hay | 3# Empower | 71.5/66 = | 1100 | 1115 | 4.75 | 5 | 5.75 | D | C− | B | YES |
| stock type | only | TLB/day | 1022 | | | | | | | | | |
| mare | | | | | | | | | | | | |
| (sorrel) | | | | | | | | | | | | |

BCS = Body Condition Score (1 is emaciated and 9 is obese. Ideal range is 5-6).
TES = Topline Evaluation Score (“A” means all three areas of the topline are ideal in muscling, “D” is all three areas are lucking in adequate muscling).

The disclosures of each and every patent, patent application, or publication cited herein are hereby incorporated by reference in their entirety. While this invention has been disclosed with reference to specific embodiments, other embodiments and variations of this invention may be devised by others skilled in the art without departing from the true spirit and scope of the invention. The appended claims are intended to be construed to include all such embodiments and variations.

1-20. (canceled)

21. A method for evaluating an attribute of a horse, comprising:
obtaining reference images of a horse's croup, loin, and wither, wherein the reference images are obtained via a 3D scanner or camera;
obtaining evaluation images of the same locations of the same horse, wherein the evaluation images are obtained via a 3D scanner or camera;
measuring a value of the attribute of the locations of the horse from the reference images;
measuring a value of the attribute of the same locations of the same horse from the evaluation images; and
comparing the values of the attribute measured in the evaluation images with the values of the attribute measured in the reference images.

22. The method of claim 21, further including determining the change in the value of the attribute.

23. The method of claim 22, further including the step of changing the horse's diet based on the change in the value of the attribute.

24. The method of claim 21, wherein the reference traces are traces of the horse's croup, loin, and/or wither generated on day 0.

25. The method of claim 21, wherein the reference traces are previous evaluation traces of the same horse's croup, loin, and/or wither generated at a time between day 0 and the time the evaluation traces are generated.

26. The method of claim 21, wherein if the area under the curve of the evaluation traces is greater than the reference traces, the musculature of the horse has improved since the time the reference traces were generated.

27. The method of claim 21, further comprising weighing the horse.

28. The method of claim 21, further comprising generating a Topline Evaluation Score (TES) based on the evaluation traces and/or the reference traces.
